(19) United States
(12) Patent Application Publication
(10) Pub. No.: US 2010/0326439 A1
(43) Pub. Date: Dec. 30, 2010

(54) INHALER

(75) Inventors: Takatoshi Tanaka, Tokyo (JP); Masaru Sugita, Tokyo (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/865,105

(22) PCT Filed: Mar. 30, 2009
(86) PCT No.: PCT/JP2009/057007

§ 371 Date: Jul. 28, 2010

(30) Foreign Application Priority Data
Mar. 31, 2008 (JP) 2008-089068
Mar. 23, 2009 (JP) 2009-070183

(51) Int. Cl. A61M 15/00

(52) U.S. Cl. 128/203.15; 128/203.12

(57) ABSTRACT

An inhaler ejects liquid droplets or powder granules into an airflow duct 5 by means of an ejection unit 6 having a plurality of ejection ports 8 so as to make the user inhale them from a mouthpiece section 7. The inhaler has a buffer space section 1 communicating with the atmosphere by way of a plurality of air intake ports 2 and is adapted to suppress turbulence of airflow in the buffer space section 1 before supplying airflow into the airflow duct 5. The inhaler reduces the quantity of ejected liquid droplets or powder granules adhering to the inner wall surface of the airflow duct and suppresses the aggravation of particle size distribution due to collisions of liquid droplets or powder granules, whichever appropriate.

### TECHNICAL FIELD

The present invention relates to an inhaler that ejects liquid droplets or powder of medicine so as to make a user inhale medicine.

### BACKGROUND ART

Inhalers that eject micro liquid droplets of medicine, utilizing the principle of ejection of ink-jet systems, into the airflow to be inhaled by a user by way of a mouthpiece so as to make the user inhale medicine have been developed (see International Publication No. WO95/01137 and International Publication No. WO02/04043). Such inhalers provide an advantage that a predetermined dose of medicine can be accurately atomized to realize a uniform particle size by means of an ink-jet system.

However, the liquid droplets that are ejected from an ejection port are more often than not very small and between 1 μm and 6 μm so that liquid droplets can easily adhere to the inner wall surface of the airflow duct.

Japanese Patent Application Laid-Open No. 2007-075227 discloses an inhaler having a straightly extending airflow duct in which no structure is arranged in view of turbulence of airflow that may arise if such a structure is arranged in the airflow duct. Then, airflow is produced in a direction perpendicular to the direction of ejection in which medicine is ejected from the ejection head. One of the opposite ends of the airflow duct is made to communicate directly with external air through an air intake port, while the other end operates as a mouthpiece.

When the airflow duct is directly provided with an air intake port as in the case of the inhalers disclosed in the above-cited three patent documents, turbulence of the airflow can easily occur when the user inhale air to produce airflow in the airflow duct by the air taken in through the air intake port. Thus, such an inhaler is apt to be externally influenced. As the turbulence of the airflow occurs in the airflow duct, medicine can adhere to the inner wall surface of the airflow duct and the particle size can vary due to collisions of liquid droplets or powder granules of medicine. Such a situation is not desirable from the viewpoint of hygiene and can waste medicine.

Additionally, with an arrangement where the direction of the airflow in the airflow duct is perpendicular to the direction of ejection of medicine, the particle size can vary due to collisions of liquid droplets or power granules of the ejected medicine. As the distribution of particle size of liquid droplets or power granules changes, the deposition sites in the user's lung change to by turn change the inhale efficiency to give rise to a situation that is by no means desirable.

### DISCLOSURE OF THE INVENTION

In view of the above-identified problem, it is therefore an object of the present invention to provide an inhaler that can reduce adhesion of medicine to the inner wall surface of the airflow duct and make the user inhale medicine with uniform particle size by suppressing turbulence of airflow in the airflow duct.

According to the present invention, the above object is achieved by providing an inhaler comprising:

an airflow duct for leading liquid or powder ejected from an ejection port to an inhalation port;

an air intake port for taking in air from the outside of the inhaler in order to produce airflow in the airflow duct at the time of inhalation;

a buffer space section for communicating with external air by way of the air intake port;

an air conduction section for leading airflow from the buffer space section into the airflow duct; and

an air supply port for supplying the airflow produced in the air conduction section to the airflow duct.

Thus, an inhaler according to the present invention can reduce turbulence of the airflow because the airflow duct is not directly provided with an air intake port. Then, as a result, both collisions of liquid droplets or powder granules being ejected and adhesion thereof to the inner wall surface of the airflow duct are suppressed so that the user can inhale a correct dose of medicine with a uniform particle size.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a schematic perspective view of an embodiment of inhaler according to the present invention.

FIG. 2A is a schematic cross sectional view of the inhaler taken along line 2A-2A in FIG. 1.

FIG. 2B is a schematic cross sectional view of the inhaler taken along line 2B-2B in FIG. 2A.

### BEST MODE FOR CARRYING OUT THE INVENTION

Preferred embodiments of the present invention will now be described in detail in accordance with the accompanying drawings.

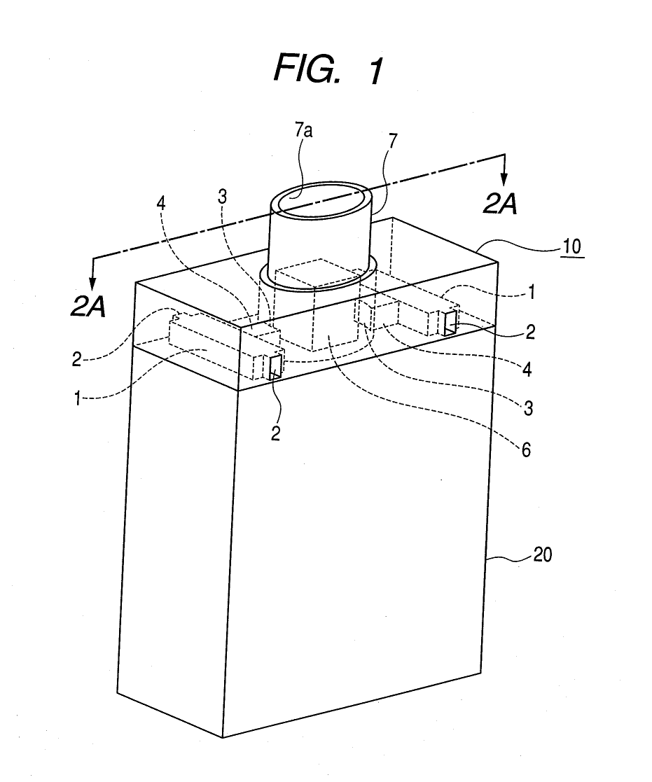

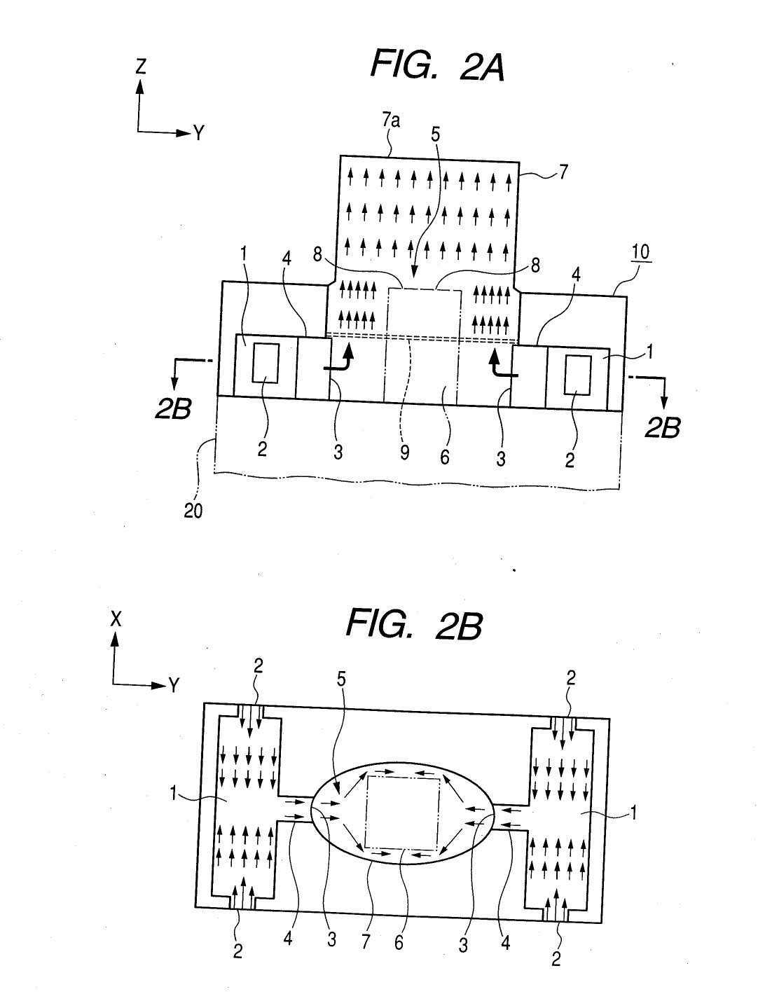

As illustrated in FIGS. 1, 2A and 2B, the inhaler of this embodiment has a box-shaped main body housing 20 and an ejection section housing 10 fitted to the top end of the main body housing 20 as illustrated in the upper part of drawings. The ejection section housing 10 by turn has an airflow duct 5 including a mouthpiece section 7 and buffer space sections 1, 1 arranged respectively at the opposite sides of the airflow duct 5.

For the purpose of the present invention, the airflow duct 5 is a duct that leads the liquid droplets or the powder granules ejected from an ejection port to an inhalation port 7a. The airflow duct is formed to show a cylindrical profile and surround an ejection unit 6. Thus, airflow takes place in the airflow duct so as to be directed in the direction in which medicine is ejected. The airflow duct 5 is not provided with any air intake port for taking in air from the outside of the inhaler at the time of inhalation. Instead, it is provided with an air supply port 3, which will be described later.

The ejection unit 6 is arranged in the airflow duct 5 such that it can eject a metered dose of medicine in the form of micro liquid droplets or powder granules highly probably having a uniform particle size at least when the inhaler is in use. The ejection unit 6 has a plurality of ejection ports 8 for ejecting liquid or powder. The ejection unit 6 may be a medicine cartridge formed by integrally combining an ejection head having ejection ports 8 and a medicine tank for containing liquid or powder.

The ejection unit 6 preferably includes an element for providing medicine with energy necessary for it to be ejected from the ejection ports 8. Typically, such an element may be an electro-thermal transducer for providing liquid medicine with thermal energy. It represents a mode of ejection of medicine that utilizes the principle of a so-called thermal ink-jet system. Alternatively, such an element may be an electro-mechanical transducer representing a mode of ejection of medicine that utilizes the principle of a so-called piezo-jet system. Still alternatively, the principle of ejection of conventional metered dose inhaler (MDI) or nebulizer or that of ejection of dry powder inhaler (DPI) may be utilized.

Air intake ports 2 are apertures for taking in air from the outside of the inhaler in order to produce airflow in the airflow duct 5 at the time of inhalation. While apertures are arranged at the surface of the ejection section housing 10, the present invention is by no means limited thereto and it is sufficient for the inhaler that such apertures are arranged at the surface of the inhaler main body.

The buffer space sections 1 communicate with the atmosphere through the air intake port 2. Therefore, as the user takes the mouthpiece section 7 between his or her teeth and inhale air, the air that is inhaled with the medicine into the body at the time of inhalation is taken into the buffer space sections 1 through the plurality of air intake ports 2. The buffer space sections 1 form spaces that are different from the space formed by the airflow duct 5.

Air conduction sections 4 and air supply ports 3 are arranged between the buffer space sections 1 and the airflow duct 5. The air conduction sections 4 are channels for leading airflow from the buffer space sections 1 into the airflow duct 5. The air supply ports 3 are exits of the channels or apertures for supplying the airflow produced in the air conduction sections 4 into the airflow duct 5.

The air taken into the buffer space sections 1 is then introduced into the airflow duct 5 from the air supply ports 3 as airflow flowing through the air conduction sections 4.

In this embodiment, the direction of the airflow produced in the airflow duct 5 runs in parallel with the direction in which medicine is ejected from the ejection unit 6 and the airflow carrying medicine is led to the mouthpiece section 7 along the lateral sides of the ejection unit 6.

Thus, the influence of fluctuations of the flow rate of the airflow at each of the air intake ports 2 on the airflow in the airflow duct 5 can be suppressed by introducing air into the airflow duct 5 by way of the buffer space sections 1 in this way rather than causing the airflow to be directly produced to the airflow duct 5 from the air intake ports 2.

Then, as a result of suppressing turbulence of the airflow in the airflow duct 5 due to the influence of fluctuations of the flow rate of the airflow at each of the air intake ports 2 by arranging buffer space sections 1, adhesion of ejected liquid droplets or powder granules to the inner wall surface of the airflow duct 5 and collision among liquid droplets or among powder granules can now be prevented.

Preferably, the air supply ports 3 are apertures arranged at the inner wall surface of the airflow duct 5. With this arrangement of the air supply ports, the space in the airflow duct 5 and the spaces in the buffer space sections 1 are separated from each other.

Preferably, the air supply ports 3 are arranged at positions remote from the inhalation port 7a relative to the ejection port 8 of the airflow duct 5. In other words, the airflow produced in the airflow duct 5 is made to pass along the lateral surfaces of the ejection unit and the ejection ports forming surface. With this arrangement, the produced airflow is directed in the direction in which the medicine is ejected so as to convey the medicine to the inhalation port 7a.

Preferably, the air supply ports 3 and the air conduction sections 4 are so arranged that the direction of airflow produced in the air conduction sections 4 is different from the direction of the airflow produced in the airflow duct 5. With this arrangement, the spaces in the buffer space sections 1 can be more clearly separated from the space in the airflow duct 5. In other words, the influence of turbulence of the airflow at the air intake ports 2 on the airflow duct 5 can be further reduced.

Preferably, the air supply ports 3 and the air conduction sections 4 are so formed that the flow rate of the airflow is higher at the air supply ports 3 and in the air conduction sections 4 than in the buffer space sections 1. In other words, the air conduction sections 4 are made narrower than the buffer space sections 1. With this arrangement, the spaces in the buffer space sections 1 can be more clearly separated from the space in the airflow duct 5. In other words, the influence of turbulence of the airflow at the air intake ports 2 on the airflow duct 5 can be further reduced.

However, it should be noted that the arrangement of the air conduction sections 4 is by no means limited to the above-described one. The air conduction sections 4 are channels through which airflow is led from the buffer space sections 1 into the airflow duct 5 and therefore the parts of the spaces in the buffer space sections 1 located close to the air supply ports 3 inevitably operate as parts of the air conduction sections 4.

Furthermore, preferably, for the purpose of the present invention, the air intake ports 2 are arranged at positions that do not give rise to any airflow in the buffer space sections 1 in a direction running in parallel with the direction of the airflow produced in the air conduction sections 4. For example, the air intake ports 2 may be arranged so as to be substantially perpendicular relative to the air supply ports 3 for supplying air into the airflow duct 5 as illustrated in FIG. 2. With this arrangement, the direction of the airflow produced in the air conduction sections 4 is substantially perpendicular relative to the direction of the airflow produced in the buffer space sections 1 so that the influence of fluctuations of the flow rate of the airflow at each of the air intake ports 2 on the airflow in the airflow duct 5 can be further suppressed.

In this embodiment, if the direction of the airflow produced in the buffer space sections 1 is X direction, the direction of the airflow produced in the air conduction sections 4 is Y direction that is perpendicular to the X direction and the direction of the airflow produced in the airflow duct 5 is Z direction that is perpendicular to both the X direction and the Y direction. Preferably, the airflow is three dimensionally drawn from the air intake port until the airflow reaches the inhalation port in this manner.

For the purpose of the present invention, each buffer space section 1 is provided with a plurality of air intake ports 2. With this arrangement, if the user closes one of the air intake ports 2 by mistake when he or she grasps the inhaler, air can be reliably drawn into the buffer space section 1.

For the purpose of the present invention, the plurality of air intake ports 2 of the same buffer space section 1 are arranged oppositely with the buffer space section 1 interposed between them. With this arrangement, the flows of air introduced through the air intake ports 2 cancel each other to reduce the average flow rate. Then, as a result, the turbulence of the airflow produced in the airflow duct 5 by way of the air conduction sections 4 can be further suppressed.

The air intake ports 2 of each of the buffer space sections 1 of this embodiment are arranged at the longitudinally oppositely disposed end facets at positions that are perfectly opposite relative to each other. However, the present invention is by no means limited thereto and it is sufficient that the air intake ports 2 are arranged at oppositely disposed ends of each buffer space section 1. For example, the air intake ports 2 may be arranged at oppositely disposed ends at positions that are offset relative to each other.

The single airflow duct 5 is provided with a pair of buffer space sections 1 in this embodiment. However, the present invention is by no means limited thereto and the single airflow duct 5 may be provided with a single buffer space section 1. Alternatively, the single airflow duct 5 may be provided with three or more than three buffer space sections 1.

The air supply ports 3 of this embodiment are directed in a direction substantially perpendicular relative to the direction of the inhalation port 7a. However, the present invention is by no means limited thereto and the air supply ports 3 may be arranged in a direction substantially running in parallel with the direction of the inhalation port 7a.

The turbulence of the airflow in the airflow duct 5 can be further reduced by arranging a flow rectifying member 9 for rectifying airflow in the space between the planes where the air supply ports 3 are arranged and the plane where the ejection ports 8 are arranged.

Then, the flow rectifying member 9 may be a lattice-shaped member that divides the cross section of the airflow duct 5, a plate-like member having a plurality of holes having a cross section smaller than that of the airflow duct 5, a fibrous member or a porous member.

The present invention is not limited to the above embodiments and various changes and modifications can be made within the spirit and scope of the present invention. Therefore, to apprise the public of the scope of the present invention, the following claims are made.

This application claims the benefit of Japanese Patent Applications No. 2008-089068, filed Mar. 31, 2008, and No. 2009-070183, filed Mar. 23, 2009, which are hereby incorporated by reference herein in their entirety.

1. An inhaler comprising:
an airflow duct for leading liquid or powder ejected from an ejection port to an inhalation port;
an air intake port for taking in air from the outside of the inhaler in order to produce airflow in the airflow duct at the time of inhalation;
a buffer space section for communicating with external air by way of the air intake port;
an air conduction section for leading airflow from the buffer space section into the airflow duct; and
an air supply port for supplying the airflow produced in the air conduction section to the airflow duct.

2. The inhaler according to claim 1, wherein
the air supply port is an aperture arranged at the inner wall surface of the airflow duct.

3. The inhaler according to claim 2, wherein
the air supply port is arranged at a position remote from the inhalation port relative to the ejection port of the airflow duct.

4. The inhaler according to Claim 1, wherein
the air supply port and the air conduction section are so arranged that the direction of airflow produced in the air conduction section is different from the direction of airflow produced in the airflow duct.

5. The inhaler according to claim 1, wherein
the air supply port and the air conduction section are so formed that the flow rate of the airflow is higher at the air supply port and in the air conduction section than in the buffer space section.

6. The inhaler according to claim 1, wherein
the air intake port is arranged at a position that does not give rise to any airflow in the buffer space section in a direction running in parallel with the direction of the airflow produced in the air conduction section.

7. The inhaler according to claim 1, wherein
the buffer space section is provided with a plurality of air intake ports and the plurality of air intake ports are arranged oppositely with the buffer space section interposed between them.

8. The inhaler according to claim 1, wherein
the airflow duct is provided with a plurality of buffer space sections.

9. The inhaler according to claim 1, further comprising:
a flow rectifying member for rectifying airflow in the space between a plane where the air supply port is arranged and a plane where the ejection port is arranged.
